(19) United States
(12) Patent Application Publication
(10) Pub. No.: US 2021/0235998 A1
(43) Pub. Date: Aug. 5, 2021

(54) Method and Apparatus for Determining the Impact of Behavior-Influenced Activities on the Health Level of a User

(71) Applicant: Biosigns Pte. Ltd., Singapore (SG)

(72) Inventor: Mattia Zanon, Zurich (CH)

(21) Appl. No.: 17/051,800

(22) PCT Filed: May 2, 2018
(86) PCT No.: PCT/CH2018/000020

§ 371 Date: Oct. 30, 2020

(51) Int. Cl. A61B 5/0205; A61B 5/024; A61B 5/11; A61B 5/00

(52) U.S. Cl. CPC A61B 5/0205; A61B 5/02444; A61B 5/1118; A61B 2562/0219; A61B 5/4809; A61B 5/02405; A61B 5/742; A61B 5/6824

(57) ABSTRACT

A method and device for determining the influence of activity of a user on a health level of a user is disclosed. It is based on measuring the heartbeat signal of the user using a heartbeat sensor (10) and calculating a health level (H) of the user depending on the heart rate variability of the user. Further, a motion signal of the user is measured using an acceleration sensor (16). Several activity parameters (Pi) are calculated. A first activity parameter (P I) depends on the amount of sleep of the user. A second activity parameter (P2) depends on the amount of steps taken by the user. Further activity parameters may be used. From this data, a fitting function is used to determine coefficients (Ci) indicative of how strongly the health level (H) depends on each of the activity parameters. The heartbeat sensor (10) can include an optical reflection sensor.

### CROSS-REFERENCE TO RELATED APPLICATION

This application is the United States national phase of International Application No. PCT/CH2018/000020 filed May 2, 2018, the disclosure of which is hereby incorporated by reference in its entirety.

### BACKGROUND OF THE INVENTION

### Field of the Invention

The invention relates to a method and an apparatus for determining the impact of behavior-influenced activities on the health level of the user.

### Description of Related Art

It is known that a person's health depends on the activities the person is involved in. For example, a good amount of sleep (as one type activity) is beneficiary to a person's health level, and so is a large number of physical steps taken each day (as another type of activity). Further types of activities may e.g. include physical exercise (beyond mere walking) or non-sleep relaxation (rest without actually sleeping).

A person's health can be characterized by a suitable parameter. One example of such a parameter is the “Accumulated resources” parameter as described in U.S. Pat. No. 7,330,752, which can be derived from heart rate variability (HRV).

### SUMMARY OF THE INVENTION

The problem to be solved by the present invention is to provide a method and an apparatus that allow to improve a person's health level.

This problem is solved by the method and apparatus of the independent claims.

Accordingly, the method for determining the impact of behavior-related activity of a user on a health level of the user comprises at least the following steps:
Measuring a heartbeat signal of the user using a heartbeat sensor: This signal e.g. allows to at least measure a user's heart rate, inter beat interval, and or the shape of individual pulses.
Calculating, from the heartbeat signal, a health level H of the user: This health level depends on the heart rate variability of the user. The heartbeat signal is used for calculating the health level H.
Measuring a motion signal: An acceleration sensor, to be worn by the user, is used to measure the motion signal.
Calculating several (i.e. two or more) activity parameters Pᵢ with i=1 . . . N and N>1. This step includes at least the calculation of a first and a second activity parameter as follows:
   a) The first activity parameter P₁ is calculated at least using the motion signal, and it is dependent on the amount of sleep, and—optionally—sleep quality, of the user in a first time period.
   b) The second activity parameter P₂ is also calculated at least from the motion signal and it is dependent on the amount of steps taken by the user in a second time period. (The first and second time periods may or may not be equal.)
Storing the health level and the activity parameters Pᵢ for a plurality of times as a dataset. This dataset describes the health level H versus the activity parameters Pᵢ at several times, in particular over a period of several days.
Fitting several function parameters aⱼ of a model function H=H(Pᵢ, aⱼ) with j=1 . . . M, in particular M≥N, to the dataset.
Deriving, from the function parameters aⱼ, activity coefficients Cᵢ, with i=1 . . . N, with Cᵢ depending on the derivative δH/δPᵢ of the model function H(Pᵢ, aⱼ) in respect to the activity parameter Pᵢ.

The invention is based on the understanding that said derivatives, and therefore the coefficients Cᵢ, describe how strongly the health level H depends on the individual activity parameters P₁. Hence, the knowledge of the activity coefficients Cᵢ allows the user to recognize how strongly their health level depends e.g. on the amount of sleep and how strongly it depends on other activities. This allows to better adjust the user's behavior in order to optimize the health level. If, for example, a strong dependence is found on sleep but a weaker one on the number of steps, the user can concentrate on getting more sleep.

Advantageously, the coefficients Cᵢ are mutually normalized. In this context, the coefficients are understood to be normalized if they are scaled with typical values (such as current values or average values) or a typical variance of the activity parameters Pᵢ. This allows to directly compare the activity coefficients Cᵢ to each other.

Examples of how to calculate mutually normalized activity coefficients Cᵢ include using normalized derivatives and/or using mutually normalized activity parameters Pᵢ.

The activity parameters Pᵢ are parameters that the user can consciously influence and thus depend on the user's behavior, such as sleep and number of steps. Some other possibilities are described in the following.

In one embodiment, the method comprises the step of calculating, using the motion and heartbeat signals, a third activity parameter P₃ that depends on the amount of non-sleep relaxation of said user in a third time period, e.g. as defined in Columns 1 and 2 of U.S. Pat. No. 73,330,752.

In another embodiment, the method comprises the step of calculating, using the motion and heartbeat signal, a fourth activity parameter P₄ depending on the amount of cardiorespiratory exercise of the user in a fourth time period. In this context, cardiorespiratory exercise is understood to be an exercise that is more strenuous than mere walking. Such cardiorespiratory exercise can e.g. include running, swimming, riding a bicycle, strenuous household chores, and in general any activity improving cardiorespiratory fitness.

In one embodiment, the steps of measuring the heartbeat signal and the motion signal and deriving said activity coefficients Cᵢ are carried out by means of a first device worn by the user. On the other hand, the activity coefficients Cᵢ are displayed on a second, separate device. This allows to reduce power consumption on the wearable device because the calculation tasks are carried out by the user-wearable device while the displaying takes place on a separate, second device.

In particular, the first device can be worn around the user's arm, in particular his upper arm, while the second device can e.g. be a smartphone, a tablet, or a computer.

The invention also relates to an apparatus for determining the influence of activity of a user on a health level of the user adapted to carry out the method described here.

In particular, the heartbeat sensor of such an apparatus comprises
A light source: The light source can be configured to send light into the user's tissue.
A light detector: The light detector can be configured to receive the light from the light source as it is reflected from the tissue.

In this case, the light source can be arranged in the center of the light detector, and the light detector can surround the light source. This design increases the sensitivity of the device. Also, it improves the accuracy of the measurement because the light detector detects light scattered in many directions. This is of importance if the tissue is non-homogeneous, e.g. due to blood vessels, muscle structure, and/or skin inhomogeneities.

### BRIEF DESCRIPTION OF THE DRAWINGS

The invention will be better understood and objects other than those set forth above will become apparent when consideration is given to the following detailed description thereof. This description makes reference to the annexed drawings, wherein:

FIG. 1 shows an apparatus for determining the influence of activity of a user on his health level,

FIG. 2 shows the user-facing side of the wearable device of FIG. 1,

FIG. 3 shows a block circuit diagram of the wearable device of FIG. 1,

FIG. 4 shows a functional block diagram of the apparatus,

FIG. 5 shows a flow chart for an activity classifier,

FIG. 6 shows a first mapping function m_{H}, and

FIG. 7 shows a second mapping function m_{P}.

### DESCRIPTION OF THE INVENTION

Apparatus:

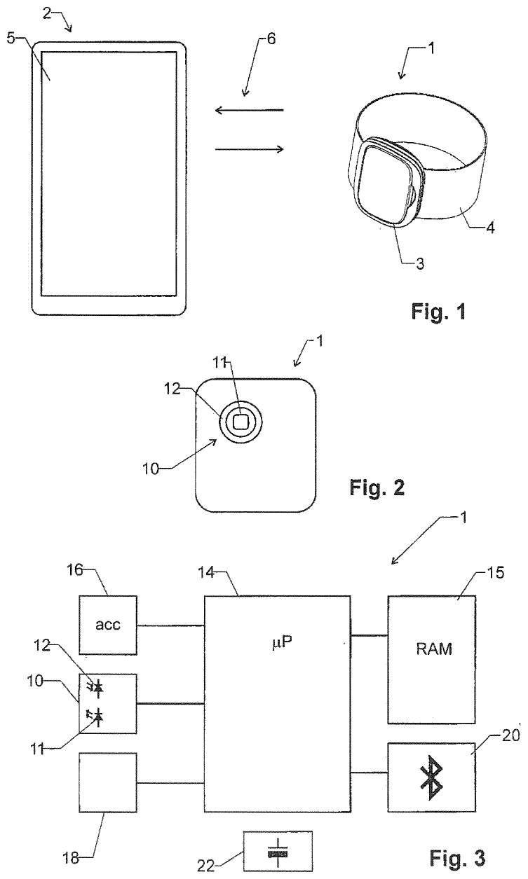

The apparatus of FIG. 1 comprises a first, user-wearable device 1 and a second device 2.

User-wearable device 1 e.g. comprises a housing 3 and a band 4 for attaching housing 3 to an arm or a leg. Advantageously, user-wearable device 1 is designed to be worn on a user's upper arm.

For example, user-wearable device 1 can basically be designed as the device described in WO2011094876.

Advantageously, user-wearable device 2 does not comprise a display. Any display function is delegated to second device 2.

Second device 2 can e.g. be a smartphone, a tablet, or a computer. It has a display 5, such as a touchscreen, for displaying information and, optionally, for receiving input from the user.

A wireless communication channel 6 may be used for communication between the first and the second devices 1, 2. For example, such communication may use the Bluetooth standard.

While second device 2 can be standard hardware running a dedicated application for displaying the results from user-worn device 1, user-worn device 1 uses dedicated hardware described in more detail in the next section.

User-Wearable Device:

FIG. 2 shows housing 3 of user-wearable device 1 from the user-facing side. Band 4 is designed such that this side can be held smugly against the user's skin.

As shown, user-wearable device may comprise an optical sensor 10 arranged on the user-facing side. It is a reflectometry sensor having a light source 11 and a light detector 12.

In one advantageous embodiment, light source 11 e.g. comprises one or more LEDs. Light detector 12 is annular. It can consist of a single, annular sensor area or a plurality of discrete light detectors 12 arranged in a circle. Light source 11 is arranged in the center of light detector 12, and light detector 12 surrounds light sensor 11. As described above, such a design is more sensitive and yields more reliable results.

Advantageously, optical sensor 10 operates at three wavelengths, one in the green spectral region, one in the red spectral region, and one in the near-infrared spectral region. However, depending on the scope of measurements required, it may also operate at a single wavelength or wavelength-region only. In the context of the present invention, it advantageously operates at a wavelength where the reflection of blood differs strongly from the reflection of other body tissue, such that blood pulses can be measured well. For example, it operates at least at one wavelength between 520 and 570 nm.

FIG. 3 shows a block diagram of an embodiment of user-wearable device 1.

A microprocessor 14 can be provided, communicating with a memory 15. Memory 15 contains software for operating the device and is also used to store data, such as calibration data as well as measured datasets, while operating the device.

Microprocessor 14 communicates with optical sensor 10 for carrying out reflection measurements on the user's tissue.

It also communicates with an accelerometer 16, such as a MEMS accelerometer. Accelerometer 16 is advantageously suited to at least measure linear acceleration in three dimensions. It can also be equipped to measure spatially resolved static acceleration, from which the device's attitude can be determined.

Device 1 may comprise one or more further sensors 18, such sensors adapted to measure the electrical impedance of the user's tissue at one or more frequencies. It also may comprise a temperature sensor.

Examples of sensors are e.g. described in US2009312615 or WO 2010/118537.

User-wearable device 1 further comprises an interface 20, such as a Bluetooth interface, for communicating with second device 2.

Device 1 is powered by a battery 22.

Functional Design:

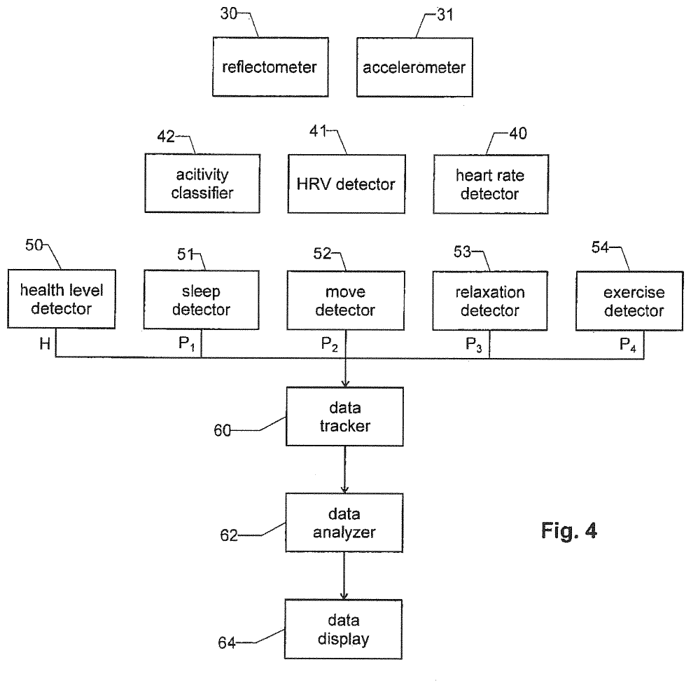

FIG. 4 shows an example of the functional design of the apparatus.

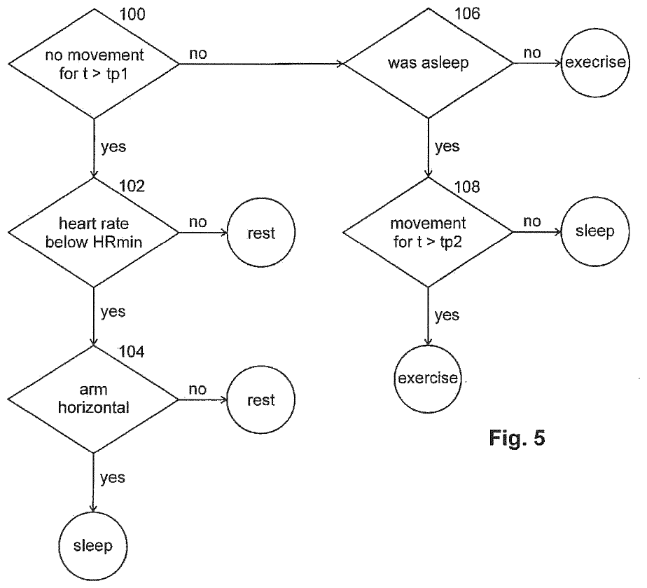

Even though the functional elements shown in that figure could each be carried out by any of the devices of the apparatus, in an advantageous embodiment, all of the elements shown in FIG. 5 except the element “data display” are implemented in the hard- and/or software of user-wearable device 1, and only “data display” is implemented by second device 2.

Basic Measurements

The top two functional blocks, reflectometer 30 and accelerometer 31, represent the basic measurements as carried out by means of optical sensor 10 and accelerometer 16.

Reflectometer 30 generates a value indicative of the current reflectivity of the user's tissue. This can e.g. be a vector-based value if measurements are carried out at several wavelengths.

This signal is termed, in the following, the “heartbeat signal” as it is indicative of the heart beat (i.e. of the amount of blood in the subcutaneous tissue).

Accelerometer 31 generates a value indicative of the current acceleration. This can e.g. be a vector-based value if acceleration is measured for several degrees of freedom.

Intermediate Data

A next set of functional elements 40-42, generates intermediate data used in one or more of the other functional elements.

A heart rate detector 40 measures the current heart rate. This value can be determined from the signal of reflectometer 30 as known to the skilled person. The value of heart rate detector 40 can e.g. describe the beats per minute or the interbeat-interval (IBI). Heart rate detector 40 can e.g. be equipped to calculate the instantaneous value of this parameter. In addition, it may be equipped to measure a time-averaged value of this parameter, e.g. over the last minute.

A heart-rate-variability detector (in the following called “HRV detector”) 41 measures heart rate variability. This value can e.g. be calculated from the interbeat interval calculated by heart rate detector 40. Methods for measuring HRV are known to the skilled person and e.g. described in https://en.wikipedia.org/wiki/Heart rate variability.

An activity classifier 42 determines the current activity of the user.

Advantageously, activity classifier 42 distinguishes between at least one, in particular between at least all, of the following states of the user:

1) Sleep

2) Rest

3) Exercise

There are various methods for distinguishing between such user states in a wearable device. The following articles describe examples of such algorithms:
Parkka, Juha, et al. “Activity classification using realistic data from wearable sensors.” IEEE Transactions on information technology in biomedicine 10.1 (2006): 119-128.
Yang, Che-Chang, and Yeh-Liang Hsu. “A review of accelerometry-based wearable motion detectors for physical activity monitoring.” Sensors 10.8 (2010): 7772-7788.
Bao, Ling, and Stephen S. Intille. “Activity recognition from user-annotated acceleration data.” International Conference on Pervasive Computing. Springer, Berlin, Heidelberg, 2004.

A simple embodiment of the steps executed by an activity classifier using the signals of the heart rate detector and the accelerometer is shown in FIG. 5.

In a first step 100, the classifier tests if there has been no movement for at least a certain time period tp1. If yes, it tests if the current heart rate (pulse rate) is below a threshold HRmin (step 102). If no, it determines that the user is at rest.

If step 102 yields yes, the user may be sleeping. In one advantageous embodiment, the activity classifier may further check for the attitude of the arm. This possible if the user-wearable device is worn on the arm and measures static acceleration in the direction along the arm. In that case, a sleeping user will typically have his arm in a horizontal position. This is particularly true for the upper arm, i.e. when the device is worn on the upper arm.

Hence, step 104 can test if the arm, in particular the upper arm, is horizontal. If not, it is assumed that the user is at rest. If yes, it is assumed that the user is asleep.

Hence, in an advantageous embodiment, the present invention comprises the step of measuring the attitude of an arm of the user, in particular an upper arm of the user, and using this attitude for determining if the user is asleep.

If, in step 100, it has been found that the user has moved within the last time period tp1, the classifier may first test, in step 106, if the user was asleep up to this point. If no, it is determined that the user is active, i.e. his state is “exercise”.

If the user has been sleeping up to this point, the classifier may test, in step 108, if the movement continues for a second time period tp2. If no, it is assumed that the user interrupted his sleep only briefly and has gone to sleep again. During this time period, and at the end of this time period, the user's state will remain “sleep”.

If, however, after step 108, the user continued moving for a time larger than tp2, activity classifier 42 decides that the user's state is active, i.e. “exercise”.

Hence, in one embodiment, the invention comprises the following steps:
Deciding, based at least on acceleration measurements by a device worn by the user, that the user is asleep: This can e.g. be based on the criteria of steps 100, 102, 104 of FIG. 5.
If the user has been found to be asleep in this way and the user starts moving, deciding that the user is not sleeping anymore, but only if moving continues for at least the time period tp2: This corresponds to step 106 and 108 of FIG. 5.

The time period tp2 is advantageously at least 1 minutes, in particular at least 5 minutes. Also, advantageously, tp2 is no more than 20 minutes, in particular no more than 5 minutes.

The time period tp1 is advantageously at least 1 minutes, in particular at least 5 minutes. Also, advantageously, tp1 is no more than 90 minutes, in particular no more than 20 minutes.

Health Level and Activity Parameters

A next set of functional elements 50-54 in FIG. 4 calculate the health level H as well as the activity parameters Pᵢ.

A health level detector 50 calculates the health level H. This is a quantity indicative of the user's health. Typically, a user will want to optimize this level, but since it is usually unclear what kind of activities are the most relevant for it, the task of optimizing it may be difficult.

In a particularly advantageous embodiment, the heart rate variability HRV is used (potentially together with other physiological parameters) for determining the health level H. Alternatively or in addition thereto, one or more parameters derived from the heartbeat signal, such as a response of the heart rate to exercise, can be used for calculating the health level H.

In a specific embodiment, the quantity Accumulated resources (in the following called A_r) as defined in column 11 of U.S. Pat. No. 7,330,752 can be used.

The A_r quantity of U.S. Pat. No. 7,330,752 can e.g. be set to a certain value, e.g. 50, at the start of the physiological day (i.e. when the user wakes up in the morning, as determined by activity classifier 42). Alternatively, it may be set to the same value as at the end of the preceding physiological day.

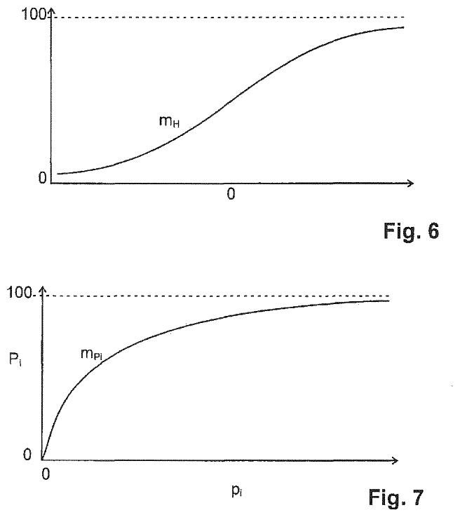

Advantageously, the health level H is obtained from A_r by mapping A_r with a monotonous mapping function m_{H} as depicted in FIG. 6, such as a sigmoid function.

A sleep detector 51 calculates a first activity parameter Pᵢ dependent on the amount and quality of sleep of the user in a first given time period.

Advantageously, the first given time period is a physiological day, against started at the start of the physiological day (i.e. when the user wakes up in the morning, as determined by activity classifier 42).

In one embodiment, the first activity parameter p₁ is increased using one or both of the following methods:
a) counting the minutes of sleep by adding those minutes for which activity classifier 42 has determined that the user is asleep;
b) using the positive values of Total resources as defined in columns 9 and 10 of U.S. Pat. No. 73,330,752 when the activity classifier 42 has determined that the user is asleep; this is one possible measure of sleep quality.

If one of these conditions are met, p₁ is increased by a given amount, e.g. 1.

At this point, the first raw activity parameter is not normalized (which is why it is called “first raw activity parameter” p₁), i.e. it may sweep over a numerical range fairly different from those of the other activity parameters. The first raw activity parameter p₁ can mapped, using a monotonous mapping function, into a predefined range, such as 0 . . . 100, in order to obtain the first activity parameter P₁. For example, a monotonous first function m_{P} as depicted in FIG. 6 can be used.

P₁ can be set to zero at the start of the physiological day (i.e. when the user wakes up in the morning, as determined by activity classifier 42) and is then increased as the day proceeds.

A move detector 52 calculates a second activity parameter P₂ at least dependent on the number of steps of the user in a second given time period.

Advantageously, the second given time period is again the physiological day starting at the start of the physiological day (i.e. when the user wakes up in the morning, as determined by activity classifier 42).

In one embodiment, a second raw activity parameter p₂ can e.g. be set to a predefined value, such as zero, at the beginning of the physiological day.

Next, a fixed value can e.g. be increased using one or both of the following methods:

a) A given value is added to the second raw activity parameter p₂ for each 30 minutes in which the user has makes at least 15 steps.

b) The parameter “Physical activity score” as described in the whitepaper Analysis of Health and Fitness-Benefits of Physical Activity Based on Heart Rate Measurements, by Firstbeat Technologies Oy, Finland, 3/2018, can be used. Whenever this value increases, a corresponding amount is added to the second raw activity parameter p₂.

If the methods a) and b) are used in combination, the values from a) are advantageously scaled such that they generate a contribution similar to those of b).

At this point, the second raw activity parameter is not normalized (therefore it is called the “second raw activity parameter” p₂), i.e. it may sweep over a numerical range fairly different from those of the other activity parameters. The second raw activity parameter p₂ can mapped, using a second monotonous mapping function, into a predefined range, such as 0 . . . 100 in order to obtain the second activity parameter P₂. For example, a monotonous function m_{P} as depicted in FIG. 6 can be used.

A relaxation detector 53 calculates a third activity parameter P₃ depending on an amount non-sleep relaxation of said user in a third time period.

The third time period may again be a physiological day.

Advantageously, a third raw activity parameter p₃ can be reset to a given value, e.g. zero, at the beginning of the physiological day.

In a simple embodiment, the third raw activity parameter can e.g. be calculated, at least in part, by adding the minutes at which the user is at rest according to activity classifier 42.

In a more refined embodiment, the third raw activity parameter p₃ is calculated by using the value Total_resources as defined in columns 9 and 10 of U.S. Pat. No. 73,330,752. p₃ is set to zero at the beginning of the physiological day. Then, at regular time intervals (e.g. once per minute), it is tested if

a) The user is not asleep as indicated by activity classifier 42 and

b) Total_resources of U.S. Pat. No. 73,330,752 is larger than zero.

If both these conditions are met, p₃ is increased by a given amount, e.g. 1.

At this point, the third raw activity parameter is not normalized, i.e. it may sweep over a numerical range fairly different from those of the other activity parameters. The third raw activity parameter p₃ can mapped, using a third monotonous mapping function, into a predefined range, such as 0 . . . 100 in order to obtain the third activity parameter P₃. For example, a monotonous function m_{P} as depicted in FIG. 6 can be used.

An exercise detector 54 calculates a fourth activity parameter P₄ depending on an amount of cardiorespiratory exercise of said user in a fourth time period.

The fourth time period may again be a physiological day.

Advantageously, a fourth raw activity parameter p₄ can be reset to a given value, e.g. zero, at the beginning of the physiological day.

In a simple embodiment, the fourth raw activity parameter can e.g. be calculated, at least in part, by adding the minutes in which the user shows large acceleration combined with a heart rate above a given threshold.

In one embodiment of the invention, the second and the fourth raw activity parameter can be distinguished by at least using the heartbeat signal from heartbeat sensor (0, in particular by comparing the heart rate calculated therefrom to the given threshold.

In a more refined embodiment, the parameter “training effect” as described in the paper “EPOC Based Training Effect Assessment by Firstbeat Technologies Oy, Finland, of March 2012 can be used which describes the effect of any physical activity on the cardiorespiratory fitness.

At this point, the fourth raw activity parameter is not normalized, i.e. it may sweep over a numerical range fairly different from those of the other activity parameters. The fourth raw activity parameter p₄ can mapped, using a fourth monotonous mapping function, into a predefined range, such as 0 . . . 100 to obtain the fourth activity parameter P₄. For example, a monotonous function m_{P} as depicted in FIG. 6 can be used.

Data Tracking, Analyzing and Displaying

Once in a given time interval, the values of the health level H as well as of the activity parameters Pᵢ are stored by a data tracker 60 as a dataset, e.g. in memory 15.

Advantageously, this occurs once each physiological day.

In particular, it occurs at the end of each physiological day, i.e. at the time immediately before activity classifier 42 determines the user to wake up.

Hence, this generates a time series dataset that shows the values of the health level H as well as of the activity parameters Pᵢ as a function of time, in particular as a function of the physiological days (e.g. one dataset is stored each physiological day).

Data tracker 60 stores at least a number of Q such datasets, e.g. over the last Q physiological days. Advantageously, for good numerical stability, the number Q is larger than, in particular at least twice as large as, the number N of activity parameters Pᵢ.

In particular, Q>10.

On the other hand, Q should not be too large in order to be able to carry out the following analysis over a reasonably recent dataset.

In particular, Q<20. Additional (older) datasets can be discarded for the following analysis.

For example, data tracker 60 may store a dataset as follows

| | | | | | |
|---|---|---|---|---|---|
| Hₜ₁ | P_{1,t1} | P_{2,t1} | P_{3,t1} | P_{4,t1} | . . . |
| Hₜ₂ | P_{1,t2} | P_{2,t2} | P_{3,t2} | P_{4,t2} | . . . |
| Hₜ₃ | P_{1,t3} | P_{2,t3} | P_{3,t3} | P_{4,t3} | . . . |
| . . . | | | | | |
| H_{tQ} | P_{1,tQ} | P_{2,tQ} | P_{3,tQ} | P_{4,tQ} | . . . |

t1, t2, t3 . . . tQ etc. are indicative of the time (e.g. the physiological day) at which the corresponding row was recorded.

Once for each new dataset, a data analyzer 62 performs a fitting process in order to determine activity coefficients Cᵢ with i=1 . . . N. The activity coefficients Cᵢ depend on the derivatives δH/δPᵢ of a model function H(Pᵢ, aⱼ) in respect to the activity parameter Pᵢ.

The model function H(Pᵢ, aⱼ) is e.g. an empirical or semi-empirical model describing how the health level H depends on the activity parameter Pᵢ. It has function parameters aⱼ, with j=1 M, in particular M≥N. The function parameters aⱼ are determined in the fitting process.

In a simple embodiment, the model function H(Pᵢ, aⱼ) is assumed to be a linear function, with the function parameters aⱼ being the coefficients attributed to the various activity parameters Pᵢ, i.e.

H(Pᵢ,aⱼ)=Σᵢ₌₁^{N}aᵢ·Pᵢ. (1)

Data analyzer 62 fits function H(Pᵢ, aⱼ) to the dataset stored by data tracker in order to obtain the function parameters aᵢ, e.g. using linear or non-linear regression analysis.

Next, data analyzer 62 derives the activity coefficients Cᵢ with i=1 . . . N. Cᵢ depends on the derivative δH/δPᵢ of the model function H(Pᵢ, aⱼ) in respect to the activity parameter Pᵢ, i.e. it is descriptive of how strongly the health level H depends on activity parameter Pᵢ.

In the linear model of Eq. (1) above, we have

δH/δPᵢ=aᵢ (2)

A knowledge of the coefficients Cᵢ allows the user to assess which of the activity parameters Pᵢ has or have a strong influence on the health level H and to change his behavior accordingly.

In order to make it easier to assess the relevance of the various activity parameters Pᵢ on the health level H, the coefficients Cᵢ are advantageously mutually normalized, i.e. they can be directly compared to each other. This can be achieved e.g. in one or more of the following ways:

a) The activity parameters Pᵢ are mutually normalized. This means that the activity parameters Pᵢ all vary over basically the same range. In the examples above, this has been achieved by mapping the raw activity parameters pᵢ to a given numerical range e.g. using functions (advantageously monotonous functions) such as depicted in FIGS. 6 and 7. In another embodiment, mutual normalization can e.g. be achieved by calculating the activity parameters Pᵢ as time values, with each activity parameter expressing the amount of time the user has spent with the given activity. In that case the partial derivatives of the health level H in respect to the activity parameters Pᵢ directly describe how much the health level H will profit when the user spends more minutes with a given activity.

b) The activity coefficients Cᵢ are derived from normalized derivatives

$$\frac{1}{Var ( {P}_{i} )} \cdot \frac{δ H}{δ {P}_{i}} \qquad ( 3 )$$

with Var(Pᵢ) being the variance of activity parameter Pᵢ. There are various algorithms for calculating variance known to the skilled person, see e.g. https://en.wikipedia.org/wiki/Algorithms_for_calculating_variance.

Eq. (3) weighs each derivative with the natural fluctuations of the given activity parameter, which again makes the derivatives mutually comparable.

In one embodiment, the coefficients Cᵢ for the examples of Eq. 1 with mutually normalized activity parameters Pᵢ could be aᵢ, or they could be a function ƒ(aᵢ) with ƒ being a monotonous function.

Data Display

The data display functional element 64 can e.g. comprise the functionality of displaying the coefficients Cᵢ on display 5 of second device 2. Alternatively, displaying can also take place on wearable device 1 and/or on any other device adapted to directly or indirectly receive data from wearable device 1 and display said data.

Notes:

While there are shown and described presently preferred embodiments of the invention, it is to be distinctly understood that the invention is not limited thereto but may be otherwise variously embodied and practiced within the scope of the following claims.

1. A method for determining an impact of behavior-related activities of a user on a health level of said user comprising the steps of
measuring a heartbeat signal of the user using a heartbeat sensor,
calculating, from said heartbeat signal, a health level H of the user depending on heart rate variability of the user,
measuring a motion signal of the user using an acceleration sensor,
calculating several activity parameters Pᵢ with i=1 N and N>1, comprising
a) calculating, using at least said motion signal, a first activity parameter P₁, wherein said first activity parameter P₁ is dependent at least on an amount of sleep of said user in a first time period,
b) calculating, using at least said motion signal, a second activity parameter P₂, wherein said second activity parameter P₂ is dependent on an amount of steps taken by said user in a second time period,
storing said health level H and said activity parameters Pᵢ for a plurality of times as a dataset of health level H versus the activity parameters Pᵢ,
fitting function parameters aⱼ of a model function H=H(Pᵢ, aⱼ) with j=1 . . . M, in particular M≥N to said dataset, and
deriving, from said function parameters aj, activity coefficients Ci with i=1 . . . N and with Ci depending on the derivative δH/δPi of said model function H(Pi, aj) in respect to the activity parameter Pi.

2. The method of claim 1, wherein said activity coefficients Cᵢ are mutually normalized.

3. The method of claim 2, wherein said activity coefficients Cᵢ are derived from normalized derivatives
$$\frac{1}{Var ( {P}_{i} )} \cdot \frac{δ H}{δ {P}_{i}}$$
with Var(Pi) being a variance of the activity parameter Pi.

4. The method of claim 1, wherein said activity parameters Pᵢ are mutually normalized.

5. The method of claim 4, wherein at least some of said activity parameters Pᵢ are obtained by mapping a raw activity parameter pᵢ into a predefined range, which predefined range is common for all said parameters Pᵢ, using a monotonous mapping function m_{Pi}.

6. The method of claim 1, further comprising the step of calculating, using said motion and heartbeat signals, a third activity parameter P₃ depending on an amount of non-sleep relaxation of said user in a third time period.

7. The method of claim 1, further comprising the step of calculating, using said motion and heartbeat signals, a fourth activity parameter P₄ depending on an amount of cardiorespiratory exercise of said user in a fourth time period.

8. The method of claim 7, further comprising the step of distinguishing contributions to said second parameter and said fourth parameter by at least using said heartbeat signal.

9. The method of claim 1, wherein said model function H(Pᵢ, aⱼ) is
$$H ( {P}_{i} , {a}_{j} ) = {\sum }_{i = 1}^{N} {a}_{i} \cdot {P}_{i} .$$

10. The method of claim 1, wherein at least some of said time periods, in particular all of said time periods, are equal to each other.

11. The method of claim 1, comprising the steps of
measuring said heartbeat signal and said motion signal and deriving said activity coefficients Cᵢ by means of a first device worn by said user and
displaying said activity coefficients Ci on a second device separate from said first device.

12. The method of claim 11, wherein said first device is worn around the user's arm, in particular the user's upper arm, and/or wherein the second device is one of a smartphone, a tablet, and a computer.

13. The method of claim 1, wherein said heartbeat signal is measured by sending light into the user's tissue and measuring an amount of reflected light.

14. The method of claim 1, further comprising the steps of
deciding, based at least on acceleration measurements by a first device worn by the user, that the user is asleep,
if the user has been found to be asleep and the user starts moving, deciding that the user is not sleeping anymore if moving continues for at least a given time period (tp2), and in particular wherein said time period (tp2) is at least 1 minutes, in particular at least 5 minutes.

15. The method of claim 1, further comprising the step of measuring an attitude of an arm of the user and using said attitude for determining if the user is asleep.

16. The method of claim 1, further comprising the steps of
measuring a heart rate variability (HRV) of the user,
calculating said health level H using said heart rate variability (HRV).

17. The method of claim 1, further comprising the step of using said heartbeat signal for calculating said first and/or second activity parameter P₁, P₂.

18. An apparatus for determining an influence of activity of a user on a health level of the user adapted to carry out the method of claim 1.

19. The apparatus of claim 18, wherein said heartbeat sensor comprises
a light source,
a light detector,
wherein said light source is arranged in a center of said light detector and wherein said light detector surrounds said light source.

20. The apparatus of claim 19, wherein said light detector is annular.
